(19) United States
(12) Patent Application Publication
(10) Pub. No.: US 2017/0071966 A1
(43) Pub. Date: Mar. 16, 2017

(54) PHARMACEUTICAL COMPOSITION

(71) Applicant: Steven BARANOWITZ, Wyncote, PA (US)

(72) Inventor: Steven BARANOWITZ, Wyncote, PA (US)

(21) Appl. No.: 15/310,496

(22) PCT Filed: May 12, 2015
(86) PCT No.: PCT/US15/30291

§ 371 Date: Nov. 11, 2016

Related U.S. Application Data
(60) Provisional application No. 61/992,459, filed on May 13, 2014.

(51) Int. Cl. A61K 31/708; A61K 31/575; A61K 31/015; B65D 75/36; A61J 1/03; B65D 1/02; B65D 75/00; A61K 9/00; A61J 1/10

(52) U.S. Cl. CPC A61K 31/708; A61K 9/006; A61K 31/575; A61K 31/015; A61J 1/10; A61J 1/035; B65D 1/02; B65D 75/002; B65D 75/36

(57) ABSTRACT

This invention relates to pharmaceutical compositions for oral administration to treat, for example, acute radiation sickness, wherein the composition comprises, for example, a first agent which is selected from the group consisting of beta-carotene, and pharmaceutically acceptable derivatives thereof; a therapeutically effective amount of a second agent which is selected from the group consisting of cholic acid, derivatives thereof, and pharmaceutically acceptable salts thereof, and at least one pharmaceutically acceptable excipient.

### CROSS-REFERENCE TO RELATED APPLICATION

This PCT application claims priority and the benefit under 35 U.S.C. §119(e) of U.S. Patent Application Ser. No. 61/992,459, filed May 13, 2014, entitled PHARMACEUTICAL COMPOSITION, the entire disclosure of which is incorporated by reference herein.

### BACKGROUND OF THE INVENTION

This invention relates to pharmaceutical compositions for oral administration to treat, for example, acute radiation sickness, wherein the composition comprises, for example, a first agent which is selected from the group consisting of beta-carotene, pharmaceutically acceptable derivatives thereof, pharmaceutically acceptable salts thereof, guanosine monophosphate, pharmaceutically acceptable derivatives thereof, pharmaceutically acceptable salts thereof, and combinations thereof; a therapeutically effective amount of a second agent which is selected from the group consisting of cholic acid, derivatives thereof, and pharmaceutically acceptable salts thereof, and at least one pharmaceutically acceptable excipient.

Most people who survive dangerous levels of radiation exposure but succumb to death several weeks afterwards die of bone marrow failure, and this is called the Hematopoietic Syndrome of the Acute Radiation Syndrome. Acute radiation syndrome (ARS), also known as radiation toxicity or radiation sickness, is an acute illness caused by irradiation of the entire body with a high dose of radiation over a short period of time. Because of differences in cell sensitivity to ionizing radiation, ARS is often divided into three components: Hematopoietic Syndrome, Gastrointestinal Syndrome, and Neurovascular Syndrome (DiCarlo, A. L., et al. 2011. Disaster Med Public Health Prep 5, Suppl 1, S32-44; and Hematopoietic Syndrome from Radiation. US Centers for Disease Control www.bt.cdc.gov/radiation/arsphysicianfactsheet.asp). The hematopoietic stem and progenitor cells of the bone marrow are normally rapidly dividing, and are highly sensitive to the effects of ionizing radiation. Hematopoietic Syndrome will develop after about 2-8 Gray (Gy) total body irradiation, and typically results in death in a few weeks. (DiCarlo, A. L., et al. 2011. Disaster Med Public Health Prep 5, Suppl 1, S32-44. Hematopoietic Syndrome from Radiation.US Centers for Disease Control, www.bt.cdc.gov/radiation/arsphysicianfactsheet.asp). There are currently no approved antidotes. Death is primarily due to radiation damaging the bone marrow cells, which are unable to produce normal amounts of white cells, resulting usually in mortality from sepsis.

It has been demonstrated in the literature that certain levels of beta-carotene when administered immediately after radiation exposure to mice, will reduce mortality and morbidity. Beta-carotene alone was demonstrated to enhance survival from radiation in mice:

(a) when administered orally as a single dose immediately after whole body radiation (Seifter et al., J. Natl. Cancer Inst. 1984 73:1167-1177; Seifter et al., Pharmac. Ther. 1988 39:357-365.)

(b) when given daily for two weeks after radiation (Beliaev et al., Vopr. Med. Khim. 1992 38:39-42, Beliaev et al., Vopr. Pitan. 1992 March-April (2) 58-61.)

(c) when given one day before whole body radiation (Al-Wandavi et al., Ann. Nutr. Metab. 2003 47:176-180)

(d) when given orally for 30 days after whole body radiation (Li, Y., Qiao, S., Zhou, L., Jiang, S., &Dong, J. (2003). Toxicology, 191(1), 53-54.) and

(e) when given for two weeks in the diet before irradiation. (Umegaki et al., Carcinogenesis 1997 18:1943-47)

The present invention provides formulations and devices for the administration of beta-carotene and/or guanosine monophosphate formulations which are novel and unique in that administration to the mouth results in rapid absorption similar to what would be achieved by intravenous injection, and can be accomplished by individuals with no medical training (e.g., not a medic, pharmacist, nurse, nor physician) to severely injured or unconscious victims, even in a mass casualty situation.

Unlike potential injectable therapies for Hematopoietic Syndrome of Acute Radiation Syndrome, which would require the presence of trained medical personnel to administer the injection, the present invention is sufficiently simple in manufacture and administration that laymen with no medical training can administer the treatment to victims of radiation exposure, even to victims who are unconscious or unresponsive. The present invention can also, of course, be administered by those with medical training. Furthermore this oral treatment is absorbed into the body and has therapeutic effect almost as quickly as an intravenous injection would. Radiation injured individuals could administer the treatment to themselves simply by putting it in their mouths and it does not require additional materials such as a drink to swallow. Patients who are incapacitated or unconscious could be given the treatment simply by the expedient method of someone placing this material in the mouth of the victim, without regard to placing the material in any specific parts of the mouth. Furthermore, while a single administration of the treatment will enhance the chances for reducing mortality and morbidity from radiation exposure, repeated administration of the invention is simple and would not result in any toxicity.

All references cited herein are incorporated herein by reference in their entireties.

### BRIEF SUMMARY OF THE INVENTION

The invention provides a pharmaceutical oral mucosal delivery composition comprising: a therapeutically effective amount of a first agent which is selected from the group consisting of beta-carotene, pharmaceutically acceptable derivatives thereof, pharmaceutically acceptable salts thereof, guanosine monophosphate, pharmaceutically acceptable derivatives thereof, pharmaceutically acceptable salts thereof, and combinations thereof a therapeutically effective amount of a second agent which is selected from the group consisting of cholic acid, derivatives thereof, and pharmaceutically acceptable salts thereof and at least one pharmaceutically acceptable excipient. The invention provides the oral mucosal delivery composition wherein the composition is provided in a sublingual or buccal dosage form. The invention provides the oral mucosal delivery composition wherein the composition comprises a pharmaceutically acceptable excipient selected from the group consisting of buffer, preservative, isotonic agent, an antioxidant, and combinations thereof. The invention provides the oral mucosal delivery composition wherein the dosage form is selected from the group consisting of a tablet, a chewing gum, a gel, a patch, a composition wherein the composition comprises the first agent in a dose from about 10 mg to lozenge, a troche, a pastille, a sachet, and a rapid disintegrating tablet. The invention provides the oral mucosal delivery about 450 mg, and the second agent in a dose from about 10 mg to about 450 mg. The invention provides the oral mucosal delivery composition wherein the first agent is beta-carotene and the first agent is present in an amount selected from the group consisting of about 75 mg, about 150 mg, about 225 mg, about 300 mg, and about 450 mg. The invention provides the oral mucosal delivery composition wherein the second agent is present in an amount selected from the group consisting of about 75 mg, about 150 mg, about 225 mg, about 300 mg, and about 450 mg. The invention provides the oral mucosal delivery composition wherein the first agent is guanosine monophosphate and the first agent is present in an amount selected from the group consisting of about 75 mg, about 150 mg, about 225 mg, about 300 mg, and about 450. The invention provides the oral mucosal delivery composition wherein the second agent is present in an amount selected from the group consisting of about 75 mg, about 150 mg, about 225 mg, about 300 mg, and about 450 mg. The invention provides the oral mucosal delivery composition of wherein the first agent is present in an amount of from 2% to 50% of the total weight of the composition. The invention provides the oral mucosal delivery composition wherein the first agent is present in an amount of from 25% to 30% of the total weight of the composition. The invention provides the oral mucosal delivery composition wherein said first agent is beta-carotene and the therapeutically effective amount of beta-carotene comprises at least about 75 mg to about 450 mg. The invention provides the oral mucosal delivery composition wherein said therapeutically effective amount of beta-carotene comprises about 240 mg. The invention provides the oral mucosal delivery composition wherein said therapeutically effective amount of guanosine monophosphate comprises at least about 75 mg to about 450 mg. The invention provides the oral mucosal delivery composition wherein said first agent is guanosine monophosphate and the therapeutically effective amount of guanosine monophosphate comprises about 240 mg. The invention provides the oral mucosal delivery composition wherein the second agent is present in an amount of from 2% to 50% of the total weight of the composition. The invention provides the oral mucosal delivery composition wherein the second agent is present in an amount of from 25% to 30% of the total weight of the composition. The invention provides the oral mucosal delivery composition wherein the concentration of the second agent is from about 0.01% to about 90% of the dry matter weight of the composition. The invention provides the oral mucosal delivery composition wherein the weight ratio of the first agent to the second agent is selected from the group consisting of about 10:1, about 5:1, about 3:1, about 2:1, about 1:1, about 1:2, about 1:3, about 1:5, and about 1:10. The invention provides the oral mucosal delivery composition wherein the composition further comprises at least one flavoring agent, artificial coloring, sweetener, lubricating agent, disintegration agent, permeation enhancer, lubricating agent, diluent, base, buffering agent, or combinations thereof.

The invention provides a method for the treatment or prevention of acute radiation syndrome (ARS) in a subject in need thereof comprising: orally administering the oral mucosal delivery composition to the subject, wherein the administration of the composition treats or prevents ARS in the subject. The invention further provides the method wherein the pharmaceutical composition is administered transmucosally through sublingual or buccal routes of delivery. The invention further provides the method wherein the oral mucosal membrane is selected from the group consisting of buccal, sublingual, and combinations thereof. The invention further provides the method wherein said first agent is beta-carotene and the therapeutically effective amount of beta-carotene comprises at least about 75 mg to about 450 mg. The invention further provides the method wherein said therapeutically effective amount of beta-carotene comprises about 240 mg. The invention further provides the method wherein said first agent is guanosine monophosphate and the therapeutically effective amount of guanosine monophosphate comprises at least about 75 mg to about 450 mg. The invention further provides the method wherein said therapeutically effective amount of guanosine monophosphate comprises about 240 mg. The invention further provides the method wherein the composition comprises the first agent in a dose from about 10 mg to about 450 mg, and the second agent in a dose from about 10 mg to about 450 mg. The invention further provides the method wherein the first agent is beta-carotene and the first agent is present in an amount selected from the group consisting of about 75 mg, about 150 mg, about 225 mg, about 300 mg, and about 450 mg. The invention further provides the method wherein the second agent is present in an amount selected from the group consisting of about 75 mg, about 150 mg, about 225 mg, about 300 mg, and about 450 mg. The invention further provides the method wherein the first agent is guanosine monophosphate and the first agent is present in an amount selected from the group consisting of about 75 mg, about 150 mg, about 225 mg, about 300 mg, and about 450 mg. The invention further provides the method wherein the second agent is present in an amount selected from the group consisting of about 75 mg, about 150 mg, about 225 mg, about 300 mg, and about 450 mg.

The invention further provides the kit for the treatment, amelioration or prevention of a condition selected from the group consisting of acute radiation syndrome (ARS), in a patient in need thereof comprising: (a) the oral mucosal delivery composition of the invention; and (b) at least one blister package; a lidded blister; a blister card or packet; a clamshell; an intravenous (IV) package, IV packette or IV container; a bottle; a metal tube; a laminate tube; a plastic tube; a dispenser; a pressurized container; a barrier container; a package; a tray or a shrink wrap, comprising the pharmaceutical composition of (a) and instructions for use of the oral mucosal delivery composition. The invention further provides the product of manufacture comprising a blister package; a lidded blister; a blister card or packet; a clamshell; an intravenous (IV) package, IV packette or IV container; a bottle; a metal tube; a laminate tube; a plastic tube; a dispenser; a pressurized container; a barrier container; a package; a tray or a shrink wrap comprising the oral mucosal delivery composition of the invention and instructions for use of the oral mucosal delivery composition. The invention further provides the a method of producing an oral mucosal delivery composition for use in mucosal delivery of the invention, the method comprising: providing a therapeutically effective amount of a first agent which is selected from the group consisting of beta-carotene, pharmaceutically acceptable derivatives thereof, pharmaceutically acceptable salts thereof, guanosine monophosphate, pharmaceutically acceptable derivatives thereof, and pharmaceutically acceptable salts thereof, and combinations thereof; providing a therapeutically effective amount of a second agent which is selected from the group consisting of cholic acid, derivatives thereof, and pharmaceutically acceptable salts thereof; providing at least one pharmaceutically acceptable excipient; and mixing the first agent, the second agent, and at least one pharmaceutically acceptable excipient to thereby produce the oral mucosal delivery composition. The invention further provides the method wherein the composition is provided in a sublingual or buccal dosage form. The invention further provides the method wherein the composition comprises a pharmaceutically acceptable excipient selected from the group consisting of buffer, preservative, isotonic agent, an antioxidant, and combinations thereof. The invention further provides the method wherein the dosage form is selected from the group consisting of a tablet, a chewing gum, a gel, a patch, a lozenge, a troche, a pastille, a sachet, and a rapid disintegrating tablet. The invention further provides the method wherein said first agent is beta-carotene and the therapeutically effective amount of beta-carotene comprises at least about 75 mg to about 450 mg. The invention further provides the method wherein said therapeutically effective amount of beta-carotene comprises about 240 mg. The invention further provides the method wherein the composition comprises the first agent in a dose from about 10 mg to about 450 mg, and the second agent in a dose from about 10 mg to about 450 mg. The invention further provides the method wherein the first agent is beta-carotene and the first agent is present in an amount selected from the group consisting of about 75 mg, about 150 mg, about 225 mg, about 300 mg, and about 450 mg. The invention further provides the method wherein the second agent is present in an amount selected from the group consisting of about 75 mg, about 150 mg, about 225 mg, about 300 mg, and about 450 mg. The invention further provides the method wherein the first agent is guanosine monophosphate and the first agent is present in an amount selected from the group consisting of about 75 mg, about 150 mg, about 225 mg, about 300 mg, and about 450 mg. The invention further provides the method wherein the second agent is present in an amount selected from the group consisting of about 75 mg, about 150 mg, about 225 mg, about 300 mg, and about 450 mg. The invention further provides the method wherein said first agent is guanosine monophosphate and the therapeutically effective amount of guanosine monophosphate comprises at least about 75 mg to about 450 mg. The invention further provides the method wherein said therapeutically effective amount of guanosine monophosphate comprises about 240 mg. The invention further provides the method wherein the weight ratio of the first agent to the second agent is selected from the group consisting of about 10:1, about 5:1, about 3:1, about 2:1, about 1:1, about 1:2, about 1:3, about 1:5, and about 1:10. The invention further provides the method wherein the composition further comprises at least one flavoring agent, artificial coloring, sweetener, lubricating agent, disintegration agent, permeation enhancer, lubricating agent, diluent, base, buffering agent, or combinations thereof.

### DETAILED DESCRIPTION OF THE INVENTION

The invention is a formulation of, for example a first agent which is selected from the group consisting of beta-carotene, pharmaceutically acceptable derivatives thereof, pharmaceutically acceptable salts thereof, guanosine monophosphate, pharmaceutically acceptable derivatives thereof, pharmaceutically acceptable salts thereof, and combinations thereof with a therapeutically effective amount of a second agent which is selected from the group consisting of cholic acid, derivatives thereof, and pharmaceutically acceptable salts thereof, and at least one pharmaceutically acceptable excipient which permits the practical and rapid administration to a mammal/human of a treatment which reduces mortality and morbidity from radiation exposure. The mixture can also be administered prophylactically in advance of anticipated radiation exposure.

The present invention relates to compositions comprising for example, beta-carotene, and pharmaceutically acceptable derivatives thereof and/or guanosine monophosphate and pharmaceutically acceptable salts and derivatives thereof; with cholic acid, derivatives thereof, and pharmaceutically acceptable salts thereof for transmucosal delivery. The compositions are suitable for sublingual, nasal, and buccal administration use, and provide for absorption of the drug across the oral and nasal mucosa.

The invention is also directed to methods of treatment comprising administering the inventive compositions by transmucosal delivery. The inventive methods may improve bioavailability relative to oral dosage forms, especially in those patients with abnormally slow gastric emptying. Such methods can involve administration of the novel compositions described herein. The methods may provide treatment for a variety of conditions amenable to amelioration by for example, beta-carotene, and pharmaceutically acceptable derivatives thereof; with cholic acid, derivatives thereof, and pharmaceutically acceptable salts thereof for transmucosal administration, without the occurrence of possible side effects associated with oral ingestion.

The present invention provides a pharmaceutical composition comprising for example, a first agent which is selected from the group consisting of beta-carotene, pharmaceutically acceptable derivatives thereof, pharmaceutically acceptable salts thereof, guanosine monophosphate, pharmaceutically acceptable derivatives thereof, pharmaceutically acceptable salts thereof, and combinations thereof; a therapeutically effective amount of a second agent which is selected from the group consisting of cholic acid, derivatives thereof, and pharmaceutically acceptable salts thereof, and at least one pharmaceutically acceptable excipient for transmucosal delivery, wherein the pharmaceutically acceptable composition is provided in a form suitable for transmucosal delivery through nasal, sublingual, or buccal administration.

When the composition of the invention is put into the mouth, rapid delivery of the treatment occurs by the following three routes: sublingual, enhanced absorption through the buccal mucosa, and enhanced absorption through the gastrointestinal tract.

The sublingual route is well-documented to absorb almost as quickly as intravenous injection, and avoids first pass metabolism. The mixture greatly enhances the absorption of the buccal mucosa, permitting its substantial surface area in the mouth to absorb almost as quickly as the sublingual route and also to avoid first pass metabolism. The mixture enhances the absorption within the gastrointestinal tract to 5 to 10 times what it would be with beta-carotene alone.

In my experience, a simple mixture of beta-carotene and/or guanosine monophosphate with sodium cholate may not remain stable over a few months. The invention involves formulations which stabilize the mixture, and also devices for the simultaneous administration of the two substances when not premixed.

Placing of a substance in the mouth results in absorption by three major routes. (1) Swallowing through the esophagus initiates digestion by the gastrointestinal tract. (2) Sublingual absorption results in rapid and substantial blood levels of the substance, approaching the effect of intravenous injection. For instance sublingual nitroglycerin provides relief within minutes from angina (cardiac pain). (3) Absorption through the buccal mucosa is the third route.

Each of these routes of absorption has its advantages and relative disadvantages. (1) Swallowing through the esophagus permits complete digestion of anything placed in the mouth, and substances in very large quantity, but it is often slower than other routes, and results in “first pass metabolism” through the liver, which may be a disadvantage. (2) Sublingual absorption is remarkably rapid and effective, and has the benefit of avoiding first pass metabolism, but there is a small surface area under the tongue for sublingual absorption to occur. (3) Buccal mucosal absorption provides a much larger surface area than does sublingual absorption, but the degree of absorption is substantially less than that of sublingual.

These and other embodiments of the invention are described herein below or are evident to persons of ordinary skill in the art based on the following disclosures.

The term “administration” of the pharmaceutically active compounds and the pharmaceutical compositions defined herein includes transmucosal application. Nasal, sublingual and buccal administration is particularly preferred in the present invention.

“Ameliorate” or “amelioration” means a lessening of the detrimental effect or severity of the disease in the subject receiving therapy, the severity of the response being determined by means that are well known in the art.

By “compatible” herein is meant that the components of the compositions which comprise the present invention are capable of being commingled without interacting in a manner which would substantially decrease the efficacy of the pharmaceutically active compound under ordinary use conditions.

The terms “effective amount” or “pharmaceutically effective amount” refer to a relatively nontoxic but sufficient amount of the agent to provide the desired biological result. That result can be reduction and/or alleviation of the signs, symptoms, or causes of a disease, such as acute radiation syndrome, or any other desired alteration of a biological system. Such amounts are described below. An appropriate “effective” amount in any individual case may be determined by one of ordinary skill in the art using routine experimentation.

As used herein, the term “excipient” means the substances used to formulate active pharmaceutical ingredients (API) into pharmaceutical formulations; in a preferred embodiment, an excipient does not lower or interfere with the primary therapeutic effect of the API. Preferably, an excipient is therapeutically inert. The term “excipient” encompasses carriers, diluents, vehicles, solubilizers, stabilizers, bulking agents, acidic or basic pH-adjusting agents and binders. Excipients can also be those substances present in a pharmaceutical formulation as an indirect or unintended result of the manufacturing process. Preferably, excipients are approved for or considered to be safe for human and animal administration, i.e., GRAS substances (generally regarded as safe). GRAS substances are listed by the Food and Drug administration in the Code of Federal Regulations (CFR) at 21 CFR 182 and 21 CFR 184, incorporated herein by reference.

As used herein, the terms “formulate” refers to the preparation of a pharmaceutical composition in a form suitable for administration to a mammalian patient, preferably a human. Thus, “formulation” can include the addition of pharmaceutically acceptable excipients, diluents, or carriers and pH adjusting agents.

“Oral mucosal delivery” refers to application of an active agent (such as a pharmaceutical composition) to one or more mucosal membranes of the oral cavity, including buccal (cheek), sublingual (under tongue), lips, gums, palates, and tongue, and passage of the active agent through the membranes covering these places and its entry into the bloodstream.

“Oral mucosal delivery vehicle” refers to a drug delivery system capable of oral mucosal delivery of at least one active agent.

The term “permeation enhancer” or “penetration enhancer” as used herein refers to an agent that improves the rate of transport of a pharmacologically active agent (e.g., beta-carotene) across the mucosal surface. Typically a penetration enhancer increases the permeability of mucosal tissue to a pharmacologically active agent. Penetration enhancers, for example, increase the rate at which the pharmacologically active agent permeates through membranes and enters the bloodstream. Enhanced permeation effected through the use of penetration enhancers can be observed, for example, by measuring the flux of the pharmacologically active agent across animal or human membranes as described in the Examples herein below. An “effective” amount of a permeation enhancer as used herein means an amount that will provide a desired increase in mucosal membranes permeability to provide, for example, the desired depth of penetration of a selected compound, rate of administration of the compound, and amount of compound delivered.

By “pharmaceutically acceptable” or “pharmacologically acceptable” is meant a material which is not biologically or otherwise undesirable, i.e., the material may be administered to an individual without causing undesirable biological effects or interacting in a deleterious manner with any of the components of the composition in which it is contained.

As used herein, a “pharmaceutically acceptable carrier” is a material that is relatively nontoxic and generally inert and does not affect the functionality of the active ingredients adversely. Examples of pharmaceutically acceptable carriers are well known and they are sometimes referred to as diluents, vehicles or excipients. The carriers may be organic or inorganic in nature. In addition, the formulation may contain additives such as flavoring agents, coloring agents, thickening or gelling agents, emulsifiers, wetting agents, buffers, stabilizers, and preservatives such as antioxidants.

The term “pharmaceutical composition” as used herein means a composition that is made under conditions such that it is suitable for administration to, for example, humans, e.g., it is made under GMP conditions and contains pharmaceutically acceptable excipients, e.g., without limitation, stabilizers, pH adjusting agents, bulking agents, buffers, carriers, diluents, vehicles, solubilizers, and binders.

A liquid carrier or vehicle can be a solvent or liquid dispersion medium comprising, for example, water, ethanol, a polyol such as glycerol, propylene glycol, or liquid polyethylene glycols and the like, vegetable oils, nontoxic glyceryl esters, and suitable mixtures thereof. The prevention of the growth of microorganisms can be accomplished by various antibacterial and antifungal agents, for example, parabens, chlorobutanol, phenol, sorbic acid, thimerosal, and the like.

As used herein, the term “subject” encompasses mammals and non-mammals. Examples of mammals include, but are not limited to, any member of the Mammalia class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. The term does not denote a particular age or sex.

As used herein, the terms “treating” or “treatment” of a disease include preventing the disease, i.e. preventing clinical symptoms of the disease in a subject that may be exposed to, or predisposed to, the disease, but does not yet experience or display symptoms of the disease; inhibiting the disease, i.e., arresting the development of the disease or its clinical symptoms, such as by suppressing or relieving the disease, i.e., causing regression of the disease or its clinical symptoms.

### Beta-Carotene

Beta-carotene is a dietary supplement and provitamin. Beta carotene is a member of a family of chemicals known as the carotenoids, which are found in many fruits and vegetables, as well as some animal products such as egg yolks. Natural extracts containing carotenoids, for example carrot extracts and red palm oil, have been used to color foods for centuries. Carotenoids were first isolated in the early 19th century, and have been synthesized for use as food colorings since the 1950s. The structure of beta carotene is:

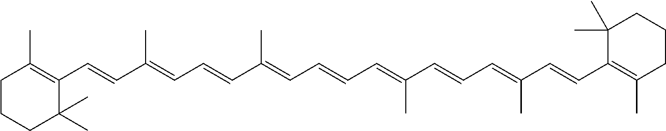 [SMILES: CC1=C(/C=C/C(C)=C/C=C/C(C)=C/C=C/C=C(C)/C=C/C=C(C)/C=C/C2=C(C)CCCC2(C)C)C(C)(C)CCC1]

Beta carotene is used in foods and beverages to provide color. For example, it is commonly used in margarine to provide its yellow color. In addition, beta carotene may be used in foods or beverages for its anti-oxidant effects. Because the body metabolizes beta carotene into Vitamin A, it is also added to foods or beverages as a nutrient.

Beta-carotene was available as a prescription drug, Solatene (Roche) for decades (until the mid-1990's) for the disease Erythropoietic Protoporphyria, and beta-carotene, is still routinely used as a first line treatment for that disease. It is typically used at doses of 180 to 300 mg per day. Extensive reviews over decades have revealed that it is safe at the highest daily dose for a lifetime (in patients with erythropoietic protoporphyria), and there is no risk of hypervitaminosis A (because the body only converts, at high beta-carotene doses, enough beta carotene to Vitamin A for its requirements and not enough to cause toxicity (Bendich A. Nutr Cancer. 1988; 11(4):207-14. Bendich A. J Nutr. 2004 January; 134(1):225S-230S.). Furthermore, careful surveillance of beta-carotene for the decades it was a prescription drug confirmed that it has no known acute toxicity.

“Derivatives”—of beta-carotene are, for example, retinol, retinoic acid, 9-cis retinoic acid, retinal and retinol.

The term “daily dosage” for example, identifies the average amount of beta-carotene administered to a patient. However, the dosage need not be administered daily. The daily dosage is merely an average dosage that a patient receives when beta-carotene is administered over a period. The daily dosage can be administered in divided portions so that the total amount administered is the daily dosage. Typically, acceptable blood levels of beta-carotene and chemically detectable changes in blood levels will be achieved after administration of beta-carotene in the prescribed amounts for several months, i.e. three to six months.

Although the safe upper limit of the amount of beta-carotene that can be administered to a human has not yet been determined, it is believed that such an upper limit is at least 1000 mg/day. Typically for a human being, a daily dosage amount will be, for example, at least about 50 mg/day of beta-carotene. Most preferably, that amount will range from about 60 mg/day to about 350 mg/day. Particularly, the dosage will be about 300 mg/day.

### Guanosine Monophosphate

Guanosine Monophosphate is a nucleotide. It is derived from guanosine, which is a nucleoside. Guanosine itself is slightly water-soluble, whereas its phosphates, including guanosine monophosphate, are highly water-soluble. Guanosine and its phosphates are components of every cell. The phosphate derivatives are dephosphorylated in the body to guanosine (Ekelman, K., and K. C. Raffaele. 1993. Disodium 5′-Guanylate and Disodium 5′-Inosinate. World Health Organization. Toxicological evaluation of certain food additives and contaminants. WHO Food Additives Series, No. 32, 1993. No. 788 on Inchem. www.inchem.org/documents/jecfa/jecmono/v32je06.htm Accessed 6/16/06.).

Methods for transdifferentiating mammalian cells that have been destabilized by radiation comprising the steps of: (a) contacting the cells with an amount of guanosine monophosphate effective to cause transdifferentiation of said cells; and (b) contacting said cells from step (a) with an amount effective for stabilization of an agent which causes stabilization of cells produced in step a. are disclosed in U.S. Pat. No. 8,575,128, incorporated by reference in its entirety herein.

In further alternative embodiments guanosine monophosphate may be substituted for with guanosine, guanosine diphosphate (GDP), guanosine triphosphate (GTP), adenosine, cytosine, thymidine, uridine and their phosphates, or catecholamines (such as norepinephrine and epinephrine).

According to the present invention, effective amounts of these reagents would broadly range between about 0.5 and about 1,000 mg/kg body weight in a recipient subject.

The term “daily dosage” for example, identifies the average amount of guanosine monophosphate administered to a patient. However, the dosage need not be administered daily. The daily dosage is merely an average dosage that a patient receives when guanosine monophosphate is administered over a period. The daily dosage can be administered in divided portions so that the total amount administered is the daily dosage. Typically, acceptable blood levels of guanosine monophosphate and chemically detectable changes in blood levels will be achieved after administration of guanosine monophosphate in the prescribed amounts for several months, i.e. three to six months.

Although the safe upper limit of the amount of guanosine monophosphate that can be administered to a human has not yet been determined, it is believed that such an upper limit is at least 1000 mg/day. Typically for a human being, a daily dosage amount will be, for example, at least about 50 mg/day of guanosine monophosphate. Most preferably, that amount will range from about 60 mg/day to about 350 mg/day. Particularly, the dosage will be about 300 mg/day.

The safety of guanosine monophosphate (as its disodium salt, Disodium Guanylate) has been extensively documented, because it is approved as a flavoring additive for food in the United States and in Europe (Kojima, K. 1974. Safety Evaluation of Disodium 5′-Inosinate, Disodium 5′-Guanylate and Disodium 5′-Ribonucleotide. Toxicology 2: 185-206.; Ekelman and Raffaele, 1993, above.). It has also been given to a few subjects in gram quantities with no known ill effects (Ekelman and Raffaele, 1993, above.).

Mice. Safety was documented in the xenograft experiment (Baranowitz, S., H. Ma, X. Li, Z. Yang, and R. M. Hoffman. 2007. Reduction Of Human Melanoma Growth By The Use Of Guanosine Monophosphate As A Transdifferentiation Agent In The Xenograft Metamouse Human Melanoma Model, LOX-GFP. J. Invest. Dermatol. 127 (Suppl. 1):S146 (Abstr. 875).) conducted for Dr Baranowitz. by a contract laboratory, Anticancer, Inc. There were three experimental groups where guanosine monophosphate was in the drinking water at 10 mg/ml, 20 mg/ml and 30 mg/ml respectively. Since these mice drink about 10 ml/day and weigh about 25 gm at the beginning of the experiment, this translates into 4000 mg/kg body weight, 8000 mg/kg, and 12000 mg/kg respectively with no obvious ill effects for this 19-day experiment. This is in line with the literature on the safety of guanosine monophosphate.

Gerbils. In a prior experiment (Baranowitz, 2001. Methods For Transdifferentiation of Body Tissues. PCT Application. International Publication Number WO 01/08691 A1. International Publication Date 8 Feb. 2001. World Intellectual Property Organization); Baranowitz, 2003 (U.S. Pat. No. 6,670,397); Baranowitz, 2005 (Australia Patent No. 782396); and Baranowitz, 2007 (U.S. Pat. No. 7,176,189 B2.), guanosine in the food 1% for 8 weeks produced no obvious deleterious effects.

Other animals. Ekelman and Raffaele (1993, above) reviewed this literature for the FDA demonstrating safety in other animal models.

Disodium guanylate, the disodium salt of guanosine monophosphate, is an FDA-approved food flavoring additive. It is a purine-based nucleotide, commonly present in foods. Disodium guanylate is listed in the Center for Food Safety and Applied Nutrition (CFSAN) database maintained by FDA under an ongoing program known as the Priority-based Assessment of Food Additives (PAFA). PAFA contains administrative, chemical, and toxicological information on over 2,000 substances directly added to food, including substances regulated by FDA as direct, “secondary” direct, color additives, GRAS, and prior-sanctioned substances. Disodium guanylate is listed in the CFSAN database as a food enhancer and is designated GRAS.

The “Everything Added to Food in the United States” (EAFUS) list of substances contains ingredients added directly to food that FDA has either approved as food additives or listed or affirmed as GRAS. Information directly applicable to disodium guanylate is provided in Table 1.

TABLE 1
Food Status Additive List for Disodium Guanylate (GRAS)

| | DOC | | CAS RN OR | |
|---|---|---|---|---|
| DOC TYPE | NUMBER | MAINTERM | OTHER CODE | REGNUM |
| ASP | 0392 | DISODIUM | 005550-12-9 | 155.120 |
| | | GUANYLATE | | 155.130 |
| | | | | 155.170 |
| | | | | 155.200 |
| | | | | 155.201 |
| | | | | 170.60 |
| | | | | 172.530 |

DOC TYPE = label;
ASP = Fully up-to-date toxicology information has been sought;
DOC NUMBER = PAFA database number containing the Food Additive Safety Profile volume containing disodium guanylate;
MAINTERM: The name of the term as recognized as CFSAN;
CAS RN or OTHER CODE: Chemical Abstract Service (CAS) registry number for the substance or a numerical code assigned to CFSAN to those substances that do not have a CAS registry number;
REGNUM = Regulation number in Title 21 of the US Code of Regulations (i.e., 21CFR 155.120) where disodium guanylate appears.

Disodium guanylate is a substance currently used as a food flavoring additive in small quantities. Its pharmacology and toxicology have therefore been well characterized, and its excellent safety record in humans has been reviewed and documented by FDA. The safety of disodium guanylate has been extensively reviewed by Ekelman and Raffaele at FDA (Ekelman, K., and K. C. Raffaele. 1993. Disodium 5′-Guanylate and Disodium 5′-Inosinate 1993; World Health Organization Toxicological evaluation of certain food additives and contaminants, WHO Food Additives Series, No. 32, 1993. No. 788).
Disodium guanylate is generally produced via fermentation to support a food grade label in compliance with the Food Chemical Codex (FCC) monograph for disodium guanylate

Disodium guanylate is a nucleotide that is found in RNA. It is an ester of phosphoric acid with the nucleoside guanosine. The chemical structure of disodium guanylate contains four chiral centers and is optically active. Disodium guanylate as shown below consists of the phosphate group, the pentose sugar ribose, and the nucleobase guanine

### Chemical Structure of Disodium Guanylate

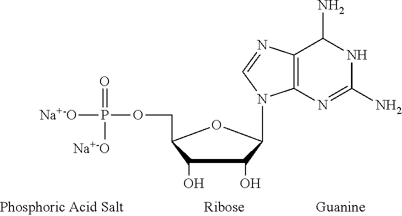

### Sodium Cholate

Sodium cholate is a bile salt used as a food additive (e.g. for cheese) or a dietary supplement. It is remarkably safe, and is categorized by FDA in the its “safest” category which is known as “Generally Regarded as Safe” (GRAS). The present invention involves the use of, for example, sodium cholate to markedly enhance rapid absorption of medically significant amounts of, for example, beta-carotene and/or guanosine monophosphate through all three routes.

Cholic acid and its derivatives are represented by Formula 1:

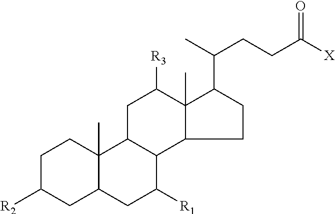

wherein R₁, R₂, and R₃ are independently hydrogen or a hydroxy group; and X is —OH, —ONa, —NH—(CH₂)ₙ—SO₃H, —NH—(CH₂)ₙ—SO₃Na, —NH—(CH₂)ₙ—SO₃K, —NH—(CH₂)ₙ—CO₂H, —NH—(CH₂)ₙ—CO₂Na, or —NH—(CH₂)ₙ—CO₂K (in which n is an integer between 1 and 3),

or Formula 2:

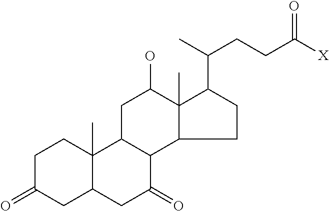

wherein X is as defined in Formula 1.

Cholic acid of the present invention has the chemical formula 3α, 7α, 12α-trihydroxycholan-24-oic acid, and is also termed ox bile extract, cholalic acid, cholalin or cholanic acid.

Examples of suitable cholic acid derivatives of the present invention include 3 α, 12α-dihydroxycholan-24-oic acid (deoxycholic acid), 3,7,12-trioxocholan-24-oic acid (dehydrocholic acid), 3α, 7α-dihydroxycholan-24-oic acid (chenodeoxycholic acid), 3α, 7β-dihydroxycholan-24-oic acid (ursodesoxycholic acid), 3 α-hydroxycholan-24-oic acid (lithocholic acid), 2-[(3α, 7α,12α-trihydroxy-24-oxocholan-24-yl)amino]ethan-esulfonic acid (taurocholic acid), 2-(3α, 7α,12α-trihydroxy-24-oxocholan-24-yl)glycine (glycocholic acid), and so on. Alkali metal salts and other derivatives that can be prepared from these cholic acid derivatives are also within the scope of the present invention. These cholic acid derivatives have a steroid structure as a common mother nucleus, and belong to steroid acids containing a carboxyl group. Acidic materials having a steroid mother nucleus which can be extracted from the bile of humans and animals, and salts with alkali metal ions thereof are also within the scope of the present invention.

A few studies in the literature report that sodium cholate can increase buccal mucosal absorption of specific substances. Senel et al, 1994 (Senel, S, A J Hoogstraate, F Spies, J C Verhoef, A Bos-van Geest, H E Junginger, and H E Bodde, Journal of controlled release 32, no. 1 (1994): 45-56.) reported that sodium cholate could increase the mucosal permeability of fluorescein isothiocyanate by 100-200 times. Nakane et al., 1996 (Nakane, Sachi, Munetaka Kakumoto, Keiji Yukimatsu, and Yie W Chien. Pharmaceutical development and technology 1, no. 3 (1996): 251-259) reported that sodium cholate increased the absorption of luteinizing hormone releasing hormone. Zhang et al., 1994 (Zhang, Jie, Suyi Niu, Charles Ebert, and Theodore H Stanley. International journal of pharmaceutics 101, no. 1 (1994): 15-22.) reported that sodium cholate increased the buccal absorption of insulin. Other researchers have demonstrated effectiveness of other bile acids or derivatives of sodium cholate to enhance absorption of the buccal mucosa. No reports have been identified in the literature which involve a combination of beta-carotene with sodium cholate to increase buccal or sublingual mucosal absorption of beta-carotene.

### Beta-Carotene and Sodium Cholate

Administration of beta-carotene and sodium cholate was demonstrated by Umegaki, 1995 to enhance gastrointestinal absorption of beta carotene. He showed that this resulted in increased absorption of beta-carotene into the bone marrow. (Umegaki et al., J. Nutr. 1995 125:3081-3086.). However, no reports have been identified in the literature which involve a combination of beta-carotene with sodium cholate to increase buccal or sublingual mucosal absorption.

Compositions of the invention include pharmaceutical compositions for oral administration to treat, for example, acute radiation sickness, wherein the composition comprises, for example, a first agent which is selected from the group consisting of beta-carotene, and pharmaceutically acceptable derivatives thereof; a therapeutically effective amount of a second agent which is selected from the group consisting of cholic acid, derivatives thereof, and pharmaceutically acceptable salts thereof, and at least one pharmaceutically acceptable excipient.

The percentage of sodium cholate in the mixture ranges from 2%-50%, and preferably in the range of 25%-30%.

The formulation will optimally reduce mortality and morbidity if administered within 72 hours of severe radiation exposure, and most optimally within 24 hours.

The stable formulations of beta-carotene can also be used for rapid administration of beta-carotene for other therapeutic purposes (e.g. to treat liver toxicity as described in Baranowitz patent U.S. Pat. No. 5,670,549, or treatment of Age Related Macular Degeneration as described in Baranowitz patent U.S. Pat. No. 5,457,135).

Exemplary Formulations of beta-carotene and sodium cholate are as set forth in Table 2:

TABLE 2

| | | Sodium | |
|---|---|---|---|
| Example | Beta-Carotene | Cholate | Ratio BC:SC |
| A | 300 mg | 300 mg | 1:1 |
| B | 300 mg | 150 mg | 2:1 |
| C | 150 mg | 150 mg | 1:1 |
| D | 150 mg | 75 mg | 2:1 |
| E | 450 mg | 450 mg | 1:1 |
| F | 450 mg | 225 mg | 2:1 |

### Guanosine Monophosphate and Sodium Cholate

Compositions of the invention include pharmaceutical compositions for oral administration to treat, for example, acute radiation sickness, wherein the composition comprises, for example, a first agent which is selected from the group consisting of guanosine monophosphate, pharmaceutically acceptable derivatives thereof, and pharmaceutically acceptable salts thereof, and a therapeutically effective amount of a second agent which is selected from the group consisting of cholic acid, derivatives thereof, and pharmaceutically acceptable salts thereof, and at least one pharmaceutically acceptable excipient.

Exemplary Formulations of guanosine monophosphate and sodium cholate are as set forth in Table 3:

TABLE 3

| | | Sodium | |
|---|---|---|---|
| Example | GM | Cholate | Ratio GM:SC |
| A | 300 mg | 300 mg | 1:1 |
| B | 300 mg | 150 mg | 2:1 |
| C | 150 mg | 150 mg | 1:1 |
| D | 150 mg | 75 mg | 2:1 |
| E | 450 mg | 450 mg | 1:1 |
| F | 450 mg | 225 mg | 2:1 |

### Pharmaceutical Compositions

Compositions of the invention include pharmaceutical compositions for oral administration to treat, for example, acute radiation sickness, wherein the composition comprises, for example, a first agent which is selected from the group consisting of beta-carotene, pharmaceutically acceptable derivatives thereof, pharmaceutically acceptable salts thereof, guanosine monophosphate, pharmaceutically acceptable derivatives thereof, pharmaceutically acceptable salts thereof, and combinations thereof; a therapeutically effective amount of a second agent which is selected from the group consisting of cholic acid, derivatives thereof, and pharmaceutically acceptable salts thereof, and at least one pharmaceutically acceptable excipient.

Exemplary dosages of the first agent include, for example, about 75 mg, about 150 mg, about 225 mg, about 300 mg, about 450 mg. Exemplary dosages of the second agent include, for example, about 75 mg, about 150 mg, about 225 mg, about 300 mg, about 450 mg.

Relative to an oral dosage form such as a tablet or capsule, buccal or sublingual delivery provides for rapid absorption, faster onset of therapeutic action and avoidance of liver or gut wall first pass metabolism. For patients who have difficulty in swallowing tablets, capsules or other solids or those who have intestinal failure, the buccal or sublingual delivery route is preferred.

Compositions for buccal administration include a first agent which is selected from the group consisting of beta-carotene, pharmaceutically acceptable derivatives thereof, pharmaceutically acceptable salts thereof, guanosine monophosphate, pharmaceutically acceptable derivatives thereof, pharmaceutically acceptable salts thereof, and combinations thereof; a therapeutically effective amount of a second agent which is selected from the group consisting of cholic acid, derivatives thereof, and pharmaceutically acceptable salts thereof, and at least one pharmaceutically acceptable excipient, for example, a solid dosage form. The solid dosage form disintegrates in an oral cavity with minimal liquid exposure and at body temperature, and ideally adheres to the body tissue of the oral cavity via direct adhesion to tissue or entrapment of the dosage form in-between the gum and inner cheek.

Compositions for sublingual administration include a first agent which is selected from the group consisting of beta-carotene, pharmaceutically acceptable derivatives thereof, pharmaceutically acceptable salts thereof, guanosine monophosphate, pharmaceutically acceptable derivatives thereof, pharmaceutically acceptable salts thereof, and combinations thereof; a therapeutically effective amount of a second agent which is selected from the group consisting of cholic acid, derivatives thereof, and pharmaceutically acceptable salts thereof, and at least one pharmaceutically acceptable excipient to form a solid dosage form. The solid dosage form disintegrates in an oral cavity at body temperature under the tongue.

The solid dosage forms can provide immediate release or controlled release or a combination thereof, wherein the dosage form disintegrates or melts in the oral cavity at body temperature with or without the aid of fluids, salivary fluids, mechanical erosion, or combinations thereof.

Alternatively, the dosage form can be sprayed into the oral cavity in the form of a solution spray or a dry powder. In another embodiment, the pharmaceutical composition can be deposited in the oral cavity of a subject in the form of a gel.

Generally, the composition can be adhesive towards the body tissue lining the patient's oral cavity. The dosage form can be, but is not limited to, tablets, a bioadhesive patch or film, gels, sponges, lozenges, hard candies, wafers, lollipops, sprays, gums, pills, pellets, spheres, combinations thereof, and other forms known to those of skill in the art.

Buccal and sublingual oral dosage forms are dosage forms that are intended to be held in the mouth or under the tongue until they have completely dissolved. Unlike most oral dosage forms in which the pharmaceutically active ingredient is delivered to the gastrointestinal tract of the patient for absorption of the drug through the stomach or intestinal epithelium, sublingual and/or buccal dosage forms are designed to release the pharmaceutically active ingredients in the mouth for absorption through oral mucosa. Buccal dosage forms are intended to be inserted into the buccal pouch (a space generally defined between a cheek and the gums) and dissolve or erode relatively slowly, whereas sublingual oral dosage forms are intended to be held under the tongue and dissolve more rapidly. As a result, buccal dosage forms, including mucoadhesive formulations, are generally formulated with excipients to optimize drug release into and through oral mucosa and to minimize release of the drug into the gastrointestinal tract. Otherwise, buccal and sublingual dosage forms are substantially similar, the differences being more a matter of degree than of kind

Sublingual and/or buccal oral dosage forms are preferred for delivering certain pharmaceutically active agents to the bloodstream. For example, many pharmaceutically active agents that are metabolized in the small intestine and/or liver (pharmaceutically active agents exhibiting what is known as “the first pass effect”) can be more effectively administered sublingually or bucally through oral mucosal tissue.

Sublingual and/or buccal oral dosage forms also may provide a faster onset of therapeutic effect and/or improved bioavailability of certain pharmaceutically active agents that can be absorbed through the oral mucosa, thereby bypassing gastrointestinal and hepatic metabolism processes. In addition, such dosage forms may be preferred for administering certain pharmaceutically active agents to achieve better patient acceptance and compliance, especially among those patients that have difficulty swallowing. Buccal and/or sublingual dosage forms may also be employed in some cases to overcome problems with pharmaceutically active agents that are poorly absorbed from the gastrointestinal tract and which may not be effectively administered transdermally, subcutaneously or intravenously.

The term “buccal delivery system” as used herein refers to a delivery system wherein an active ingredient is provided for absorption across one or more membranes in the mouth, including the buccal mucosa, buccal gingiva, mucous membrane of the tongue, sublingual membrane and the soft palate. The term encompasses all suitable dosage forms capable of manufacture using a normal dry powder process and compression using a standard tabletting machine.

Reference to an “active ingredient” includes a therapeutic or prophylactic agent, drug, pro-drug, drug complex, drug intermediate, diagnostic agent, enzyme, medicine, plant extract, herbal concoction, phytochemical, proteins, antibody, nanobody, antibody fragment, antibody directed enzyme pro-drug therapy (ADEPT), bioactive compound, nutraceutical or dietary supplement.

The term “matrix” as used herein refers to a solid or semi-solid monolithic material containing one or more dissolved or dispersed active ingredients closely associated with a surrounding, rate-controlling heterogeneous material where the active ingredient(s) are released when the matrix is placed in direct contact with a moist diffusion membrane. The solid or semi-solid monolithic material can include a range of materials known in the art of pharmaceutical drug delivery to emulsify, solubilize, complex or deliver any biologically active lipophilic or hydrophilic compound across a membrane.

The person skilled in the art will know which polyethylene glycol (PEG) is suitable to provide the desired pharmacokinetics for the delivery system. For example, the choice of PEG will be related to whether zero or first order release is desired. In a particularly preferred embodiment, the base is PEG 1450.

The polyethylene glycol can be used in the form of a PEG-fatty acid ester having surfactant properties. Examples of suitable PEG-fatty acid esters include PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, PEG-1450, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, a poloxamer, and mixtures thereof.

A person skilled in the art will understand that amount of the suspending agent is sufficient to improve the texture and consistency of the delivery system. Suitable examples of such suspending agents are those in the gum-yielding plant group such as tetragonolobus, Acacia glaucophylla, Acacia abyssinica, Acacia nilotica, Acacia gummifera and Acacia arabica. Other suitable suspending agents include silica gel and suspension polymers such as kollidon, cremaphor, kollicoat, solutol and ludipress.

The person skilled in the art will understand that the flowing agent (also known as lubricant) is present in an amount sufficient for the prevention of adhesion, especially during the manufacturing process. A suitable example of a flowing agent is magnesium stearate.

The buccal delivery system comprises a sufficient amount of a sweetener to improve the organoleptic properties of the dosage form. Examples of suitable sweeteners include sucrose, sucralose; zinc gluconate; ethyl maltitol; glycine; acesulfame-K; aspartame; saccharin; fructose; xylitol; honey; corn syrup, golden syrup, misri, spray dried licorice root; glycerrhizine; dextrose; sodium gluconate; stevia powder; glucono delta-lactone; ethyl vanillin; vanillin; normal and high-potency sweeteners or syrups or salts thereof. Preferably, a high-intensity sweetener selected from the group consisting of aspartame, sucralose, and acesulfame-K is used.

In another aspect, the present invention provides a method of manufacturing a dosage formulation capable of delivering one or more active ingredients across one or more membranes within the buccal cavity.

The buccal delivery system may further comprise one or more other pharmaceutically acceptable carriers and/or excipients, such as but not limited to binding agents, flavoring agents, colouring agents, solubility enhancers, disintegrants, fillers, proteins, co-factors, emulsifiers, and solubilizing or complexing agents. In a preferred embodiment, these excipients will improve delivery of the active ingredient across a membrane. Suitable excipients will be known to those skilled in the art. One typical example of an emulsifier which may be suitable is tocopherol polyethylene glycol 1000 succinate (TPGS). Examples of complexing agents are compounds containing amine groups or other nitrogen functional groups such as amino acids, proteins, amine functional sterols and phospholipids containing amine functional groups. Suitable surfactants may be amphoteric, zwitterionic, or cationic. Preferred complexing agents of this type include water-soluble cationic polymers with a quaternary ammonium functional group on the polymer backbone and water-soluble, cationic guar (jaguar gums).

In a preferred embodiment, the buccal delivery system comprises a binding and gelling agent such as hydroxypropyl methocellulose.

In another embodiment, the buccal delivery system further comprises a colouring agent which may be a dye or a pigment. Suitable colouring agents are well known in the art and include curcumin, carotenoids, sunset yellow, tartrazine, indigo dyes, quino-phthalene dyes and triphenyl methane dyes.

In a preferred embodiment, the buccal delivery system further comprises a flavoring agent for improving organoleptic properties. Suitable flavoring agents are well known in the art and include almond oil; babassu oil; borage oil; blackcurrant seed oil; canola oil; castor oil; coconut oil; corn oil; cottonseed oil; evening primrose oil; grapeseed oil; groundnut oil; mustard seed oil; olive oil; palm oil; palm kernel oil; peanut oil; grapeseed oil; safflower oil; sesame oil; shark liver oil; soybean oil; sunflower oil; hydrogenated castor oil; hydrogenated coconut oil; hydrogenated palm oil; hydrogenated soybean oil; hydrogenated vegetable oil; hydrogenated cottonseed and castor oil; partially hydrogenated soybean oil; soy oil; glyceryl tricaproate; glyceryl tricaprylate; glyceryl tricaprate; glyceryl triundecanoate; glyceryl trilaurate; glyceryl trioleate; glyceryl trilinoleate; glyceryl trilinolenate; glyceryl tricaprylate/caprate; glyceryl tricaprylate/caprate/laurate; glyceryl tricaprylate/caprate/linoleate; glyceryl tricaprylate/caprate/stearate; saturated polyglycolized glycerides; linoleic glycerides; caprylic/capric glycerides; modified triglycerides; fractionated triglycerides; safrole, vanillin, citric acid, malic acid and phosphoric acid or salts and/or mixtures thereof.

In an alternative embodiment, the buccal dosage forms are useful as sustained release compositions. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period.

Formulations and solid oral dosage forms of this invention include disintegrating agents (disintegrants) such as sodium carboxylmethyl cellulose, crospovidone and the like in amounts sufficient to achieve a desirable and efficient disintegration rate that optimizes absorption of the pharmaceutically active agent, minimizes patient discomfort and inconvenience, or achieves a desired balance of absorption efficiency and reduced discomfort and/or inconvenience. Examples of suitable amounts of disintegrating agents, such as crospovidone (e.g., Polyplasdone XL, ISP) may range from about 2 to about 50% based on the weight of the direct compression formulation and/or oral dosage form.

In some embodiments, direct compression formulations and solid oral dosage forms of this invention may further comprise other optional ingredients as desired, including natural and/or artificial sweeteners such as aspartam, taste-masking agents and/or flavorants such as menthol, and colorants (e.g., red iron oxide dye). Glidants, lubricants such as magnesium stearate, and other processing aids may be employed as needed or desired to facilitate handling and/or compression into tablets or other oral dosage forms.

### Film

In one embodiment of the present invention, there is provided a film dosage composition including: a polymeric carrier matrix; a therapeutically effective amount of a first agent which is selected from the group consisting of beta-carotene, pharmaceutically acceptable derivatives thereof, pharmaceutically acceptable salts thereof, guanosine monophosphate, pharmaceutically acceptable derivatives thereof, and pharmaceutically acceptable salts thereof, and combinations thereof; a therapeutically effective amount of a second agent which is selected from the group consisting of cholic acid, derivatives thereof, and pharmaceutically acceptable salts thereof, and at least one pharmaceutically acceptable excipient; and a buffer in an amount to provide a pH of the composition of a value sufficient to optimize absorption of the active ingredients.

It will be understood that the term “film” includes thin films and sheets, in any shape, including rectangular, square, or other desired shape. The films described herein may be any desired thickness and size such that it may be placed into the oral cavity of the user. For example, the films may have a relatively thin thickness of from about 0.1 to about 10 mils, or they may have a somewhat thicker thickness of from about 10 to about 30 mils. For some films, the thickness may be even larger, i.e., greater than about 30 mils. Films may be in a single layer or they may be multi-layered, including laminated films.

Oral dissolving films generally fall into three main classes: fast dissolving, moderate dissolving and slow dissolving. Fast dissolving films generally dissolve in about 1 second to about 30 seconds in the mouth. Moderate dissolving films generally dissolve in about 1 to about 30 minutes in the mouth, and slow dissolving films generally dissolve in more than 30 minutes in the mouth. Fast dissolving films may consist of low molecular weight hydrophilic polymers (i.e., polymers having a molecular weight between about 1,000 to 9,000, or polymers having a molecular weight up to 200,000). In contrast, slow dissolving films generally have high molecular weight polymers (i.e., having a molecular weight in the millions).

Moderate dissolving films tend to fall in between the fast and slow dissolving films. Moderate dissolving films dissolve rather quickly, but also have a good level of mucoadhesion. Moderate dissolving films are also flexible, quickly wettable, and are typically non-irritating to the user. For the instant invention, it is preferable to use films that fall between the categories of fast dissolving and moderate dissolving. Such moderate dissolving films provide a quick enough dissolution rate, most desirably between about 1 minute and about 20 minutes, while providing an acceptable mucoadhesion level such that the film is not easily removable once it is placed in the oral cavity of the user.

The films used in the pharmaceutical products may be produced by a combination of at least one polymer and a solvent, optionally including other fillers known in the art. The solvent may be water, a polar organic solvent including, but not limited to, ethanol, isopropanol, acetone, or any combination thereof. In some embodiments, the solvent may be a non-polar organic solvent, such as methylene chloride. The film may be prepared by utilizing a selected casting or deposition method and a controlled drying process. For example, the film may be prepared through controlled drying processes, which include application of heat and/or radiation energy to the wet film matrix to form a visco-elastic structure, thereby controlling the uniformity of content of the film. Such processes are described in more detail in commonly assigned U.S. application Ser. No. 10/074,272, filed on Feb. 14, 2002, and published as U.S. Patent Publication No. 2003/0107149 A1, the contents of which are incorporated herein by reference in their entirety. Alternatively, the films may be extruded as described in commonly assigned U.S. application Ser. No. 10/856,176, filed on May 28, 2004, and published as U.S. Patent Publication No. 2005/0037055 A1, the contents of which are incorporated herein by reference in their entirety.

The polymer included in the films may be water-soluble, water-swellable, water-insoluble, or a combination of one or more either water-soluble, water-swellable or water-insoluble polymers. The polymer may include cellulose or a cellulose derivative. Specific examples of useful water-soluble polymers include, but are not limited to, polyethylene oxide, pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, xanthan gum, tragancanth gum, guar gum, acacia gum, arabic gum, polyacrylic acid, methylmethacrylate copolymer, carboxyvinyl copolymers, starch, gelatin, and combinations thereof. Specific examples of useful water-insoluble polymers include, but are not limited to, ethyl cellulose, hydroxypropyl ethyl cellulose, cellulose acetate phthalate, hydroxypropyl methyl cellulose phthalate and combinations thereof. For higher dosages, it may be desirable to incorporate a polymer that provides a high level of viscosity as compared to lower dosages.

As used herein the phrase “water-soluble polymer” and variants thereof refer to a polymer that is at least partially soluble in water, and desirably fully or predominantly soluble in water, or absorbs water. Polymers that absorb water are often referred to as being water-swellable polymers. The materials useful with the present invention may be water-soluble or water-swellable at room temperature and other temperatures, such as temperatures exceeding room temperature. Moreover, the materials may be water-soluble or water-swellable at pressures less than atmospheric pressure. Desirably, the water-soluble polymers are water-soluble or water-swellable having at least 20 percent by weight water uptake. Water-swellable polymers having a 25 or greater percent by weight water uptake are also useful. In some embodiments, films formed from such water-soluble polymers may be sufficiently water-soluble to be dissolvable upon contact with bodily fluids.

Other polymers useful for incorporation into the films include biodegradable polymers, copolymers, block polymers and combinations thereof. It is understood that the term “biodegradable” is intended to include materials that chemically degrade, as opposed to materials that physically break apart (i.e., bioerodable materials). Among the known useful polymers or polymer classes which meet the above criteria are: poly(glycolic acid) (PGA), poly(lactic acid) (PLA), polydioxanes, polyoxalates, poly(.alpha.-esters), polyanhydrides, polyacetates, polycaprolactones, poly(orthoesters), polyamino acids, polyaminocarbonates, polyurethanes, polycarbonates, polyamides, poly(alkyl cyanoacrylates), and mixtures and copolymers thereof. Additional useful polymers include, stereopolymers of L- and D-lactic acid, copolymers of bis(p-carboxyphenoxy)propane acid and sebacic acid, sebacic acid copolymers, copolymers of caprolactone, poly(lactic acid)/poly(glycolic acid)/polyethyleneglycol copolymers, copolymers of polyurethane and (poly(lactic acid), copolymers of polyurethane and poly(lactic acid), copolymers of .alpha.-amino acids, copolymers of .alpha.-amino acids and caproic acid, copolymers of .alpha.-benzyl glutamate and polyethylene glycol, copolymers of succinate and poly(glycols), polyphosphazene, polyhydroxy-alkanoates and mixtures thereof. Binary and ternary systems are contemplated.

Other specific polymers useful include those marketed under the Medisorb and Biodel trademarks. The Medisorb materials are marketed by the Dupont Company of Wilmington, Del. and are generically identified as a “lactide/glycolide co-polymer” containing “propanoic acid, 2-hydroxy-polymer with hydroxy-polymer with hydroxyacetic acid.” Four such polymers include lactide/glycolide 100 L, believed to be 100% lactide having a melting point within the range of 338° F.-347° F. (170 C); lactide/glycolide 100 L, believed to be 100% glycolide having a melting point within the range of 437° F.-455° F. (225 C-235 C); lactide/glycolide 85/15, believed to be 85% lactide and 15% glycolide with a melting point within the range of 338° F.-347° F. (170 C-175 C); and lactide/glycolide 50/50, believed to be a copolymer of 50% lactide and 50% glycolide with a melting point within the range of 338° F.-347° F. (170 C-175 C).

The Biodel materials represent a family of various polyanhydrides which differ chemically.

Although a variety of different polymers may be used, it is desired to select polymers that provide mucoadhesive properties to the film, as well as a desired dissolution and/or disintegration rate. In particular, the time period for which it is desired to maintain the film in contact with the mucosal tissue depends on the type of active contained in the composition. Some actives may only require a few minutes for delivery through the mucosal tissue, whereas other actives may require up to several hours or even longer. Accordingly, in some embodiments, one or more water-soluble polymers, as described above, may be used to form the film. In other embodiments, however, it may be desirable to use combinations of water-soluble polymers and polymers that are water-swellable, water-insoluble and/or biodegradable, as provided above. The inclusion of one or more polymers that are water-swellable, water-insoluble and/or biodegradable may provide films with slower dissolution or disintegration rates than films formed from water-soluble polymers alone. As such, the film may adhere to the mucosal tissue for longer periods or time, such as up to several hours, which may be desirable for delivery of certain active components.

Desirably, the individual film dosage has a small size, which is between about 0.5-1 inch by about 0.5-1 inch. Most preferably, the film dosage is about 0.75 inches.times.0.5 inches. The film dosage should have good adhesion when placed in the buccal cavity or in the sublingual region of the user. Further, the film dosage should disperse and dissolve at a moderate rate, most desirably dispersing within about 1 minute and dissolving within about 3 minutes. In some embodiments the film dosage may be capable of dispersing and dissolving at a rate of between about 1 to about 1.5 minutes.

For instance, in some embodiments, the films may include polyethylene oxide alone or in combination with a second polymer component. The second polymer may be another water-soluble polymer, a water-swellable polymer, a water-insoluble polymer, a biodegradable polymer or any combination thereof. Suitable water-soluble polymers include, without limitation, any of those provided above. In some embodiments, the water-soluble polymer may include hydrophilic cellulosic polymers, such as hydroxypropyl cellulose and/or hydroxypropylmethyl cellulose. In accordance with some embodiments, polyethylene oxide may range from about 20% to 100% by weight in the polymer component, more specifically about 30% to about 70% by weight, and even more specifically about 40% to about 60% by weight. In some embodiments, one or more water-swellable, water-insoluble and/or biodegradable polymers also may be included in the polyethylene oxide-based film. Any of the water-swellable, water-insoluble or biodegradable polymers provided above may be employed. The second polymer component may be employed in amounts of about 0% to about 80% by weight in the polymer component, more specifically about 30% to about 70% by weight, and even more specifically about 40% to about 60% by weight.

The molecular weight of the polyethylene oxide also may be varied. In some embodiments, high molecular weight polyethylene oxide, such as about 4 million, may be desired to increase mucoadhesivity of the film. In some other embodiments, the molecular weight may range from about 100,000 to 900,000, more specifically from about 100,000 to 600,000, and even more specifically from about 100,000 to 300,000. In some embodiments, it may be desirable to combine high molecular weight (600,000 to 900,000) with low molecular weight (100,000 to 300,000) polyethylene oxide in the polymer component.

A variety of optional components and fillers also may be added to the films. These may include, without limitation: surfactants; plasticizers; polyalcohols; anti-foaming agents, such as silicone-containing compounds, which promote a smoother film surface by releasing oxygen from the film; thermo-setting gels such as pectin, carageenan, and gelatin, which help in maintaining the dispersion of components; inclusion compounds, such as cyclodextrins and caged molecules; coloring agents; and flavors. In some embodiments, more than one active components may be included in the film.

Additives may be included in the films. Examples of classes of additives include excipients, lubricants, buffering agents, stabilizers, blowing agents, pigments, coloring agents, fillers, bulking agents, sweetening agents, flavoring agents, fragrances, release modifiers, adjuvants, plasticizers, flow accelerators, mold release agents, polyols, granulating agents, diluents, binders, buffers, absorbents, glidants, adhesives, anti-adherents, acidulants, softeners, resins, demulcents, solvents, surfactants, emulsifiers, elastomers and mixtures thereof. These additives may be added with the active ingredient(s).

Useful additives include, for example, gelatin, vegetable proteins such as sunflower protein, soybean proteins, cotton seed proteins, peanut proteins, grape seed proteins, whey proteins, whey protein isolates, blood proteins, egg proteins, acrylated proteins, water-soluble polysaccharides such as alginates, carrageenans, guar gum, agar-agar, xanthan gum, gellan gum, gum arabic and related gums (gum ghatti, gum karaya, gum tragancanth), pectin, water-soluble derivatives of cellulose: alkylcelluloses hydroxyalkylcelluloses and hydroxyalkylalkylcelluloses, such as methylcelulose, hydroxymethylcellulose, hydroxyethylcellulose, hydroxypropylcellulose, hydroxyethylmethylcellulose, hydroxypropylmethylcellulose, hydroxybutylmethylcellulose, cellulose esters and hydroxyalkylcellulose esters such as cellulose acetate phthalate (CAP), hydroxypropylmethylcellulose (HPMC); carboxyalkylcelluloses, carboxyalkylalkylcelluloses, carboxyalkylcellulose esters such as carboxymethylcellulose and their alkali metal salts; water-soluble synthetic polymers such as polyacrylic acids and polyacrylic acid esters, polymethacrylic acids and polymethacrylic acid esters, polyvinylacetates, polyvinylalcohols, polyvinylacetatephthalates (PVAP), polyvinylpyrrolidone (PVP), PVY/vinyl acetate copolymer, and polycrotonic acids; also suitable are phthalated gelatin, gelatin succinate, crosslinked gelatin, shellac, water-soluble chemical derivatives of starch, cationically modified acrylates and methacrylates possessing, for example, a tertiary or quaternary amino group, such as the diethylaminoethyl group, which may be quaternized if desired; and other similar polymers.

Such extenders may optionally be added in any desired amount desirably within the range of up to about 80%, desirably about 3% to 50% and more desirably within the range of 3% to 20% based on the weight of all film components.

Further additives may flow agents and opacifiers, such as the oxides of magnesium aluminum, silicon, titanium, etc. desirably in a concentration range of about 0.02% to about 3% by weight and desirably about 0.02% to about 1% based on the weight of all film components.

Further examples of additives are plasticizers which include polyalkylene oxides, such as polyethylene glycols, polypropylene glycols, polyethylene-propylene glycols, organic plasticizers with low molecular weights, such as glycerol, glycerol monoacetate, diacetate or triacetate, triacetin, polysorbate, cetyl alcohol, propylene glycol, sorbitol, sodium diethylsulfosuccinate, triethyl citrate, tributyl citrate, and the like, added in concentrations ranging from about 0.5% to about 30%, and desirably ranging from about 0.5% to about 20% based on the weight of the polymer.

There may further be added compounds to improve the texture properties of the starch material such as animal or vegetable fats, desirably in their hydrogenated form, especially those which are solid at room temperature. These fats desirably have a melting point of 50° C. or higher. Preferred are tri-glycerides with C₁₂-, C₁₄-, C₁₆-, C₁₈-, C₂₀- and C₂₂-fatty acids. These fats can be added alone without adding extenders or plasticizers and can be advantageously added alone or together with mono- and/or di-glycerides or phosphatides, especially lecithin. The mono- and di-glycerides are desirably derived from the types of fats described above, i.e. with C₁₂-, C₁₄-, C₁₆-, C₁₈-, C₂₀- and C₂₂-fatty acids.

The total amounts used of the fats, mono-, di-glycerides and/or lecithins are up to about 5% and preferably within the range of about 0.5% to about 2% by weight of the total film composition.

It further may be useful to add silicon dioxide, calcium silicate, or titanium dioxide in a concentration of about 0.02% to about 1% by weight of the total composition. These compounds act as flow agents and opacifiers.

Lecithin is one surface active agent for use in the films described herein. Lecithin may be included in the feedstock in an amount of from about 0.25% to about 2.00% by weight. Other surface active agents, i.e. surfactants, include, but are not limited to, cetyl alcohol, sodium lauryl sulfate, the Spans® and Tweens® which are commercially available from ICI Americas, Inc. Ethoxylated oils, including ethoxylated castor oils, such as Cremophor EL which is commercially available from BASF, are also useful. Carbowax® is yet another modifier which is very useful in the present invention. Tweens® or combinations of surface active agents may be used to achieve the desired hydrophilic-lipophilic balance (“HLB”). The present invention, however, does not require the use of a surfactant and films or film-forming compositions of the present invention may be essentially free of a surfactant while still providing the desirable uniformity features of the present invention.

Other ingredients include binders which contribute to the ease of formation and general quality of the films. Non-limiting examples of binders include starches, pregelatinize starches, gelatin, polyvinylpyrrolidone, methylcellulose, sodium carboxymethylcellulose, ethylcellulose, polyacrylamides, polyvinyloxoazolidone, and polyvinylalcohols.

Further potential additives include solubility enhancing agents, such as substances that form inclusion compounds with active components. Such agents may be useful in improving the properties of very insoluble and/or unstable actives. In general, these substances are doughnut-shaped molecules with hydrophobic internal cavities and hydrophilic exteriors. Insoluble and/or instable actives may fit within the hydrophobic cavity, thereby producing an inclusion complex, which is soluble in water. Accordingly, the formation of the inclusion complex permits very insoluble and/or instable actives to be dissolved in water. A particularly desirable example of such agents are cyclodextrins, which are cyclic carbohydrates derived from starch. Other similar substances, however, are considered well within the scope of the present invention.

Suitable coloring agents include food, drug and cosmetic colors (FD&C), drug and cosmetic colors (D&C), or external drug and cosmetic colors (Ext. D&C). These colors are dyes, their corresponding lakes, and certain natural and derived colorants. Lakes are dyes absorbed on aluminum hydroxide.

Other examples of coloring agents include known azo dyes, organic or inorganic pigments, or coloring agents of natural origin. Inorganic pigments are preferred, such as the oxides or iron or titanium, these oxides, being added in concentrations ranging from about 0.001 to about 10%, and preferably about 0.5 to about 3%, based on the weight of all the components.

Flavors may be chosen from natural and synthetic flavoring liquids. An illustrative list of such agents includes volatile oils, synthetic flavor oils, flavoring aromatics, oils, liquids, oleoresins or extracts derived from plants, leaves, flowers, fruits, stems and combinations thereof. A non-limiting representative list of examples includes mint oils, cocoa, and citrus oils such as lemon, orange, grape, lime and grapefruit and fruit essences including apple, pear, peach, grape, strawberry, raspberry, cherry, plum, pineapple, apricot or other fruit flavors.

Other useful flavorings include aldehydes and esters such as benzaldehyde (cherry, almond), citral i.e., alphacitral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanol (green fruit), and 2-dodecenal (citrus, mandarin), combinations thereof and the like.

The sweeteners may be chosen from the following non-limiting list: glucose (corn syrup), dextrose, invert sugar, fructose, and combinations thereof, saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; sugar alcohols such as sorbitol, mannitol, xylitol, and the like. Also contemplated are hydrogenated starch hydrolysates and the synthetic sweetener 3,6-dihydro-6-methyl-1-1-1,2,3-oxathiazin-4-one-2,2-dioxide, particularly the potassium salt (acesulfame-K), and sodium and calcium salts thereof, and natural intensive sweeteners, such as Lo Han Kuo. Other sweeteners may also be used.

Anti-foaming and/or de-foaming components may also be used with the films. These components aid in the removal of air, such as entrapped air, from the film-forming compositions. Such entrapped air may lead to non-uniform films. Simethicone is one particularly useful anti-foaming and/or de-foaming agent. The present invention, however, is not so limited and other anti-foam and/or de-foaming agents may suitable be used.

In another embodiment, the composition comprises antioxidant(s), for example, tocopherol and derivatives, ascorbic acid and derivatives, butylated hydroxyanisole, butylated hydroxytoluene, fumaric acid, malic acid, propyl gallate, sodium metabisulfite and derivatives, is a concentration of about 0.01 to about 5 weight percent; more preferred is a concentration of about 0.1 to about 0.5 weight percent, depending on the type of antioxidant used, as known by the one skilled in the art.

In another embodiment, the composition comprises buffering agent(s), for example, carbonate buffers, citrate buffers, phosphate buffers, acetate buffers, hydrochloric acid, lactic acid, tartaric acid, inorganic and organic bases, is a concentration of about 1 to about 10 weight percent, more preferred is a concentration of about 2 to about 5 weight percent, depending on the type of buffering agent(s) used, as known by the one skilled in the art. The preferred concentration range of said buffering agents are those enabling design of compositions having a pH close to the physiologic pH of the mucosal membranes, between about pH 2.0 and about pH 10.0, preferably between about pH 3.0 and pH 7.0. Concentrations of the buffering agent(s) may vary, however, as known by the one skilled in the art. The buffering agent may replace up to 100% of the water amount within the composition.

The transmucosal pharmaceutical formulation of the present invention may also further include preservatives such as benzalkonium chloride and derivatives, benzoic acid, benzyl alcohol and derivatives, bronopol, parabens, centrimide, chlorhexidine, cresol and derivatives, imidurea, phenol, phenoxyethanol, phenylethyl alcohol, phenylmercuric salts, thimerosal, sorbic acid and derivatives. The preservative is present from about 0.01 to about 10% w/w depending on the type of compound used, as known by the one skilled in the art.

The transmucosal pharmaceutical formulation of the present invention may also further include humectants, sequestering agents, moisturizers, surfactants, emollients, colorants, fragrances, flavors, or any combination thereof.

In some embodiments, the transmucosal dosage form is a liquid formulation, comprising: a therapeutically effective amount of a first agent which is selected from the group consisting of beta-carotene, pharmaceutically acceptable derivatives thereof, pharmaceutically acceptable salts thereof, guanosine monophosphate, pharmaceutically acceptable derivatives thereof, and pharmaceutically acceptable salts thereof, and combinations thereof; a therapeutically effective amount of a second agent which is selected from the group consisting of cholic acid, derivatives thereof, and pharmaceutically acceptable salts thereof, and at least one pharmaceutically acceptable excipient, aqueous solvent; and a polar organic solvent, wherein the polar organic solvent is present in an amount sufficient to enhance the solubility of the active ingredients thereof in the water.

In one embodiment, a gel formulation of the present invention comprises a first agent which is selected from the group consisting of beta-carotene, pharmaceutically acceptable derivatives thereof, pharmaceutically acceptable salts thereof, guanosine monophosphate, pharmaceutically acceptable derivatives thereof, and pharmaceutically acceptable salts thereof, and combinations thereof; a therapeutically effective amount of a second agent which is selected from the group consisting of cholic acid, derivatives thereof, and pharmaceutically acceptable salts thereof, and at least one pharmaceutically acceptable excipient, of between about 0.01 to about 5 weight percent. The primary vehicle may comprise between about 10 to about 60 weight percent of water, between about 30 to about 70 weight percent ethanol, between about 15 and about 60 weight percent of a 10:1 to 1:10 (weight to weight) mixture of diethylene glycol mono ethyl ether and propylene glycol, and between about 0.1 and about 2 weight percent of lauryl alcohol, myristyl alcohol, oleyl alcohol, lauric acid, myristic acid, or oleic acid. The primary vehicle may be gellified with between about 0.5 and about 5 weight percent of hydroxypropylcellulose. The apparent pH of the gel is between about pH 2.0 and about pH 10.0, or preferably between about pH 3.0 and pH 7.0.

In addition, the transmucosal delivery system of the pharmaceutical composition can include a buffer to maintain the pH of the formulation and a pharmaceutically acceptable thickening agent. The pharmaceutical composition can further include one or more pharmaceutical excipients and even further include a pharmaceutically acceptable preservative.

The buffer of the transmucosal delivery system can be selected from the group including acetate, citrate, prolamine, carbonate and phosphate buffers.

The thickening agent of the transmucosal delivery system can be selected from the group including methyl cellulose, xanthan gum, carboxymethyl cellulose, hydroxypropyl cellulose, carbomer, polyvinyl alcohol, alginates, acacia, chitosans and combinations thereof.

The formulation may further comprise a sweetener suitable for sublingual and buccal delivery systems. The sweetener may be, but is not limited to, mannitol, saccharin or saccharin sodium. The formulation may further comprise a flavoring agent. Preferably, the flavoring agent is menthol. The formulation may further comprise a thickening agent. The thickening agent may be, but is not limited to, methyl cellulose, xanthan gum, carboxymethyl cellulose, hydroxypropyl cellulose, carbomer, polyvinyl alcohol, alginates, acacia, chitosans and combinations thereof.

The formulation may further comprise a humectant suitable for nasal delivery system. The humectant may be, but not limited to, sorbitol, glycerol, mineral oil, vegetable oil and combinations thereof.

In some embodiments, the transmucosal carrier of the transmucosal dosage unit is preferably an aqueous solution. Further, the aqueous solution can be selected from the group including aqueous gels, aqueous suspensions, aqueous liposomal dispersions, aqueous emulsions, aqueous microemulsions, aqueous nanoparticles and combinations thereof.

Alternatively, the carrier of the transmucosal dosage unit is a nonaqueous solution. The non-aqueous solution can be selected from a group including non-aqueous gels, non-aqueous suspensions, non-aqueous liposomal dispersions, nonaqueous emulsions, non-aqueous microemulsions, non-aqueous nanoparticles and combinations thereof.

The carrier of the transmucosal dosage unit can also be a combination of an aqueous solution and a non-aqueous solution. The formulation may be partially pressurized. Alternatively, the carrier of the transmucosal dosage unit is a powder formulation.

The powder formulation can be selected from, but not limited to, a simple powder mixtures, powder microspheres, coated powder microspheres, liposomal dispersions and combinations thereof. Preferably, the powder formulation is simple powder mixture.

In some embodiments the oral transmucosal dosage form is chosen from: a chewing gum, a patch, a gel, a lozenge, a tablet, a troche, a pastille, a sachet, and a rapid disintegrating tablet.

The formulations of the present invention may be provided in a unit dose container(s). Such containers typically comprise inner and outer surfaces, wherein the formulation of the present invention is contained by the inner surface of the container. In selected embodiments, the container is a packet or a vial, and the inner surface of the container may further comprise a liner. For example, in one embodiment, the container is a flexible, foil packet and the liner is a polyethylene liner. Alternatively, or in addition, the formulations of the present invention may be provided in a multiple dose container(s). Such multiple dose containers typically comprise inner and outer surfaces, wherein the gel for pharmaceutical drug delivery is contained by the inner surface of the container. Multiple dose containers may, for example, dispenses fixed or variable metered doses. Multiple dose containers may, for example, be a stored-energy metered dose pump or a manual metered dose pump.

### Packaging/Treatment Kits

The present invention relates to a kit for conveniently and effectively carrying out the methods in accordance with the present invention. Such kits may be suited for the delivery of, for example, solid oral forms such as tablets or capsules. Such a kit may include a number of unit dosages. Such kits can include a means for containing the dosages oriented in the order of their intended use. An example of a means for containing the dosages in the order of their intended uses is a card. An example of such a kit is a “blister pack”. Blister packs are well known in the packaging industry and are widely used for packaging pharmaceutical unit dosage forms. If desired, the blister can be in the form of a childproof blister, i.e., a blister that is difficult for a child to open, yet can be readily opened by an adult. If desired, a memory aid can be provided, for example in the form of numbers, letters, or other markings or with a calendar feature and/or calendar insert, designating the days and the sections of a day in the treatment schedule in which the dosages can be administered, such as an AM dose is packaged with a “mid day” and a PM dose.; or an AM dose is packaged with a PM dose. Alternatively, placebo dosages, or vitamin or dietary supplements, either in a form similar to or distinct from the pharmaceutical active dosages, can be included.

In one aspect, the package, kit or container comprises a “blister package” (also called a blister pack, or bubble pack). In one aspect, the blister package consists two or more separate compartments: Am dosage of this invention, and PM dosage of this invention, or mid-day dosage of this invention. This blister package is made up of two separate material elements: a transparent plastic cavity shaped to the product and its blister board backing. These two elements are then joined together with a heat sealing process which allows the product to be hung or displayed. Exemplary types of “blister packages” include: Face seal blister packages, gang run blister packages, mock blister packages, interactive blister packages, slide blister packages.

Blister packs, clamshells or trays are forms of packaging used for goods; thus, the invention provides for blister packs, clamshells or trays comprising a composition (e.g., a (the multi-ingredient combination of drugs of the invention) combination of active ingredients) of the invention. Blister packs, clamshells or trays can be designed to be non-reclosable, so consumers can tell if a package has already opened. They are used to package for sale goods where product tampering is a consideration, such as the pharmaceuticals of the invention. In one aspect, a blister pack of the invention comprises a moulded PVC base, with raised areas (the “blisters”) to contain the tablets, pills, etc. comprising the combinations of the invention, covered by a foil laminate. Tablets, pills, etc. are removed from the pack either by peeling the foil back or by pushing the blister to force the tablet to break the foil. In one aspect, a specialized form of a blister pack is a strip pack.

In one aspect, a blister pack also comprises a method of packaging where the compositions comprising combinations of ingredients of the invention are contained in-between a card and a clear PVC. The PVC can be transparent so the item (pill, tablet, geltab, etc.) can be seen and examined easily; and in one aspect, can be vacuum-formed around a mould so it can contain the item snugly and have room to be opened upon purchase. In one aspect, the card is brightly colored and designed depending on the item (pill, tablet, geltab, etc.) inside, and the PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed pills, tablets, geltabs, etc., the card has a perforated window for access. In one aspect, more secure blister packs, e.g., for items such as pills, tablets, geltabs, etc. of the invention are used, and they can comprise of two vacuum-formed PVC sheets meshed together at the edges, with the informative card inside.

In one aspect, blister packaging comprises at least two components (e.g., is a multi-ingredient combination of drugs of the invention): a thermoformed “blister” which houses the product (e.g., a pharmaceutical combination of the invention), and then a “blister card” that is a printed card with an adhesive coating on the front surface. During the assembly process, the blister component, which is most commonly made out of PVC, is attached to the blister card using a blister machine. This machine introduces heat to the flange area of the blister which activates the glue on the card in that specific area and ultimately secures the PVG blister to the printed blister card. The thermoformed PVG blister and the printed blister card can be as small or large. Conventional blister packs can also be sealed (e.g., using an AERGO 8 DUO®, SCA Consumer Packaging, Inc., DeKalb, Ill.) using regular heat seal tooling. This alternative aspect, using heat seal tooling, can seal common types of thermoformed packaging.

As discussed herein, the products of manufacture of the invention can comprise the packaging of the therapeutic drug combinations of the invention, alone or in combination, as “blister packages” or as a plurality of packettes, including as lidded blister packages, lidded blister or blister card or packets, or a shrink wrap.

In one aspect, laminated aluminum foil blister packs are used, e.g., for the preparation of drugs designed to dissolve immediately in the mouth of a patient. This exemplary process comprises having the drug combinations of the invention prepared as an aqueous solution(s) which are dispensed (e.g., by measured dose) into an aluminum (e.g., alufoil) laminated tray portion of a blister pack. This tray is then freeze-dried to form tablets which take the shape of the blister pockets. The alufoil laminate of both the tray and lid fully protects any highly hygroscopic and/or sensitive individual doses. In one aspect, the pack incorporates a child-proof peel open security laminate. In one aspect, the system give tablets an identification mark by embossing a design into the alufoil pocket that is taken up by the tablets when they change from aqueous to solid state. In one aspect, individual ‘push-through’ blister packs/packettes are used, e.g., using hard temper aluminum (e.g., alufoil) lidding material. In one aspect, hermetically-sealed high barrier aluminum (e.g., alufoil) laminates are used. In one aspect, any of the invention's products of manufacture, including kits or blister packs, use foil laminations and strip packs, stick packs, sachets and pouches, peelable and non-peelable laminations combining foil, paper, and film for high barrier packaging.

In one embodiment of the invention, the composition is contained within one suitable container, such as a dropper, a jar, or a tube with a suitable small orifice size, such as an extended tip tube, made of any pharmaceutically suitable material. The formulations according to embodiments of the invention can be filled and packaged into a plastic squeeze bottle or tube. Optionally, an applicator can be provided in or attached to the container, or separately from the container.

Kits are also contemplated as being used in certain aspects of the present invention. For instance, a composition of the present invention can be included in a kit. A kit can include a container. Containers can include a bottle, a metal tube, a laminate tube, a plastic tube, a dispenser, a pressurized container, a barrier container, a package, a compartment, or other types of containers such as injection or blow-molded plastic containers into which the dispersions or compositions or desired bottles, dispensers, or packages are retained. The kit and/or container can include indicia on its surface. The indicia, for example, can be a word, a phrase, an abbreviation, a picture, or a symbol.

The containers can dispense a pre-determined amount of a composition. In other embodiments, the container can be squeezed (e.g., metal, laminate, or plastic tube) to dispense a desired amount of the composition. The composition can be dispensed as a spray, foam, an aerosol, a liquid, a gel, a fluid, or a semi-solid. The containers can have spray, pump, or squeeze mechanisms. A kit can also include instructions for using the kit and/or compositions. Instructions can include an explanation of how to apply, use, and maintain the compositions.

Other means for containing said unit dosages can include bottles and vials, wherein the bottle or vial comprises a memory aid, such as a printed label for administering said unit dosage or dosages. The label can also contain removable reminder stickers for placement on a calendar or dayminder to further help the patient to remember when to take a dosage or when a dosage has been taken.

While the invention has been described in detail and with reference to specific examples thereof, it will be apparent to one skilled in the art that various changes and modifications can be made therein without departing from the spirit and scope thereof.

What is claimed is:

1. A pharmaceutical oral mucosal delivery composition comprising:
a therapeutically effective amount of a first agent which is selected from the group consisting of beta-carotene, pharmaceutically acceptable derivatives thereof, pharmaceutically acceptable salts thereof, guanosine monophosphate, pharmaceutically acceptable derivatives thereof, pharmaceutically acceptable salts thereof, and combinations thereof;
a therapeutically effective amount of a second agent which is selected from the group consisting of cholic acid, derivatives thereof, and pharmaceutically acceptable salts thereof; and
at least one pharmaceutically acceptable excipient.

2. The oral mucosal delivery composition of claim 1 wherein the composition is provided in a sublingual or buccal dosage form.

3. The oral mucosal delivery composition of claim 1, wherein the composition comprises a pharmaceutically acceptable excipient selected from the group consisting of buffer, preservative, isotonic agent, an antioxidant, and combinations thereof.

4. The oral mucosal delivery composition of claim 1 wherein the dosage form is selected from the group consisting of a tablet, a chewing gum, a gel, a patch, a lozenge, a troche, a pastille, a sachet, and a rapid disintegrating tablet.

5. The oral mucosal delivery composition of claim 1 wherein the composition comprises the first agent in a dose from about 10 mg to about 450 mg, and the second agent in a dose from about 10 mg to about 450 mg.

6. The oral mucosal delivery composition of claim 1 wherein the first agent is beta-carotene and the first agent is present in an amount selected from the group consisting of about 75 mg, about 150 mg, about 225 mg, about 300 mg, and about 450 mg.

7. The oral mucosal delivery composition of claim 1 wherein the second agent is present in an amount selected from the group consisting of about 75 mg, about 150 mg, about 225 mg, about 300 mg, and about 450 mg.

8. The oral mucosal delivery composition of claim 1 wherein the first agent is guanosine monophosphate and the first agent is present in an amount selected from the group consisting of about 75 mg, about 150 mg, about 225 mg, about 300 mg, and about 450.

9. The oral mucosal delivery composition of claim 1 wherein the second agent is present in an amount selected from the group consisting of about 75 mg, about 150 mg, about 225 mg, about 300 mg, and about 450 mg.

10. The oral mucosal delivery composition of claim 1 wherein the first agent is present in an amount of from 2% to 50% of the total weight of the composition.

11. The oral mucosal delivery composition of claim 1 wherein the first agent is present in an amount of from 25% to 30% of the total weight of the composition.

12. The oral mucosal delivery composition of claim 1 wherein said first agent is beta-carotene and the therapeutically effective amount of beta-carotene comprises at least about 75 mg to about 450 mg.

13. The oral mucosal delivery composition of claim 12 wherein said therapeutically effective amount of beta-carotene comprises about 240 mg.

14. The oral mucosal delivery composition of claim 12 wherein said therapeutically effective amount of guanosine monophosphate comprises at least about 75 mg to about 450 mg.

15. The oral mucosal delivery composition of claim 1 wherein said first agent is guanosine monophosphate and the therapeutically effective amount of guanosine monophosphate comprises about 240 mg.

16. The oral mucosal delivery composition of claim 1 wherein the second agent is present in an amount of from 2% to 50% of the total weight of the composition.

17. The oral mucosal delivery composition of claim 1 wherein the second agent is present in an amount of from 25% to 30% of the total weight of the composition.

18. The oral mucosal delivery composition of claim 1 wherein the concentration of the second agent is from about 0.01% to about 90% of the dry matter weight of the composition.

19. The oral mucosal delivery composition of claim 1 wherein the weight ratio of the first agent to the second agent is selected from the group consisting of about 10:1, about 5:1, about 3:1, about 2:1, about 1:1, about 1:2, about 1:3, about 1:5, and about 1:10.

20. The oral mucosal delivery composition of claim 1 wherein the composition further comprises at least one flavoring agent, artificial coloring, sweetener, lubricating agent, disintegration agent, permeation enhancer, lubricating agent, diluent, base, buffering agent, or combinations thereof.

21. A method for the treatment or prevention of acute radiation syndrome (ARS) in a subject in need thereof comprising:
orally administering the oral mucosal delivery composition of claim 1 to the subject, wherein the administration of the composition treats or prevents ARS in the subject.

22. The method of claim 21 wherein the pharmaceutical composition is administered transmucosally through sublingual or buccal routes of delivery.

23. The method of claim 21, wherein the oral mucosal membrane is selected from the group consisting of buccal, sublingual, and combinations thereof.

24. A method as defined in claim 21, wherein said first agent is beta-carotene and the therapeutically effective amount of beta-carotene comprises at least about 75 mg to about 450 mg.

25. A method as defined in claim 24, wherein said therapeutically effective amount of beta-carotene comprises about 240 mg.

26. A method as defined in claim 21, wherein said first agent is guanosine monophosphate and the therapeutically effective amount of guanosine monophosphate comprises at least about 75 mg to about 450 mg.

27. A method as defined in claim 26, wherein said therapeutically effective amount of guanosine monophosphate comprises about 240 mg.

28. A method as defined in claim 21, wherein the composition comprises the first agent in a dose from about 10 mg to about 450 mg, and the second agent in a dose from about 10 mg to about 450 mg.

29. A method as defined in claim 21, wherein the first agent is beta-carotene and the first agent is present in an amount selected from the group consisting of about 75 mg, about 150 mg, about 225 mg, about 300 mg, and about 450 mg.

30. A method as defined in claim 21, wherein the second agent is present in an amount selected from the group consisting of about 75 mg, about 150 mg, about 225 mg, about 300 mg, and about 450 mg.

31. A method as defined in claim 21, wherein the first agent is guanosine monophosphate and the first agent is present in an amount selected from the group consisting of about 75 mg, about 150 mg, about 225 mg, about 300 mg, and about 450 mg.

32. A method as defined in claim 21, wherein the second agent is present in an amount selected from the group consisting of about 75 mg, about 150 mg, about 225 mg, about 300 mg, and about 450 mg.

33. A kit for the treatment, amelioration or prevention of a condition selected from the group consisting of acute radiation syndrome (ARS), in a patient in need thereof comprising:
(a) the oral mucosal delivery composition of claim 1; and
(b) at least one blister package; a lidded blister; a blister card or packet; a clamshell; an intravenous (IV) package, IV packette or IV container; a bottle; a metal tube; a laminate tube; a plastic tube; a dispenser; a pressurized container; a barrier container; a package; a tray or a shrink wrap,
comprising the pharmaceutical composition of (a) and instructions for use of the oral mucosal delivery composition.

34. A product of manufacture comprising a blister package; a lidded blister; a blister card or packet; a clamshell; an intravenous (IV) package, IV packette or IV container; a bottle; a metal tube; a laminate tube; a plastic tube; a dispenser; a pressurized container; a barrier container; a package; a tray or a shrink wrap comprising the oral mucosal delivery composition of claim 1 and instructions for use of the oral mucosal delivery composition.

35. A method of producing an oral mucosal delivery composition for use in mucosal delivery of claim 1, the method comprising:
providing a therapeutically effective amount of a first agent which is selected from the group consisting of beta-carotene, pharmaceutically acceptable derivatives thereof, pharmaceutically acceptable salts thereof, guanosine monophosphate, pharmaceutically acceptable derivatives thereof, and pharmaceutically acceptable salts thereof, and combinations thereof;
providing a therapeutically effective amount of a second agent which is selected from the group consisting of cholic acid, derivatives thereof, and pharmaceutically acceptable salts thereof;
providing at least one pharmaceutically acceptable excipient; and
mixing the first agent, the second agent, and at least one pharmaceutically acceptable excipient to thereby produce the oral mucosal delivery composition.

36. The method of claim 35 wherein the composition is provided in a sublingual or buccal dosage form.

37. The method of claim 35, wherein the composition comprises a pharmaceutically acceptable excipient selected from the group consisting of buffer, preservative, isotonic agent, an antioxidant, and combinations thereof.

38. The method of claim 35 wherein the dosage form is selected from the group consisting of a tablet, a chewing gum, a gel, a patch, a lozenge, a troche, a pastille, a sachet, and a rapid disintegrating tablet.

39. A method as defined in claim 35, wherein said first agent is beta-carotene and the therapeutically effective amount of beta-carotene comprises at least about 75 mg to about 450 mg.

40. A method as defined in claim 39, wherein said therapeutically effective amount of beta-carotene comprises about 240 mg.

41. A method as defined in claim 39, wherein the composition comprises the first agent in a dose from about 10 mg to about 450 mg, and the second agent in a dose from about 10 mg to about 450 mg.

42. A method as defined in claim 35, wherein the first agent is beta-carotene and the first agent is present in an amount selected from the group consisting of about 75 mg, about 150 mg, about 225 mg, about 300 mg, and about 450 mg.

43. A method as defined in claim 35, wherein the second agent is present in an amount selected from the group consisting of about 75 mg, about 150 mg, about 225 mg, about 300 mg, and about 450 mg.

44. A method as defined in claim 35, wherein the first agent is guanosine monophosphate and the first agent is present in an amount selected from the group consisting of about 75 mg, about 150 mg, about 225 mg, about 300 mg, and about 450 mg.

45. A method as defined in claim 35, wherein the second agent is present in an amount selected from the group consisting of about 75 mg, about 150 mg, about 225 mg, about 300 mg, and about 450 mg.

46. A method as defined in claim 35, wherein said first agent is guanosine monophosphate and the therapeutically effective amount of guanosine monophosphate comprises at least about 75 mg to about 450 mg.

47. A method as defined in claim 45, wherein said therapeutically effective amount of guanosine monophosphate comprises about 240 mg.

48. The method of claim 35 wherein the weight ratio of the first agent to the second agent is selected from the group consisting of about 10:1, about 5:1, about 3:1, about 2:1, about 1:1, about 1:2, about 1:3, about 1:5, and about 1:10.

49. The method of claim 35 wherein the composition further comprises at least one flavoring agent, artificial coloring, sweetener, lubricating agent, disintegration agent, permeation enhancer, lubricating agent, diluent, base, buffering agent, or combinations thereof.
